(19) United States
(12) Patent Application Publication
(10) Pub. No.: US 2005/0234513 A1
(43) Pub. Date: Oct. 20, 2005

(54) Implantable neuronal networks

(76) Inventors: Phillip Alexander, San Diego, CA (US); Paul George, Boston, MA (US); Amir Nashat, Newton, MA (US); Robert Langer, Newton, MA (US); David LaVan, Hamden, CT (US); Alvin Lyckman, Brighton, MA (US); Mriganka Sur, Cambridge, MA (US); Nathan Wilson, Cambridge, MA (US)

Correspondence Address: CHOATE, HALL & STEWART LLP, TWO INTERNATIONAL PLACE, BOSTON, MA, 02110, US

(21) Appl. No.: 10/964,886

(22) Filed: Oct. 14, 2004

Related U.S. Application Data
(60) Provisional application No. 60/517,421, filed on Nov. 5, 2003.

(51) Int. Cl. A61N 1/00

(52) U.S. Cl. 607/2

(57) ABSTRACT

Method and apparatus for regenerating function in the nervous system. The method includes implanting in a central or peripheral nervous system environment neurons programmed for a selected function in the implant environment. The neurons are programmed using a multi-electrode device or micro-patterning. A suitable implantable neuronal network construct includes a conductive polymer substrate and neurons programmed for a selected function residing on the substrate.

This application claims priority to Provisional Patent Application Ser. No. 60/517,421 filed Nov. 5, 2003, the contents of which are incorporated herein by reference.

### BACKGROUND OF THE INVENTION

This invention relates to implantable neuronal networks and more specifically to such networks programmed for a selected function in the implant environment.

Many neurological disorders afflicting millions of people are related to a loss of neuronal function. Such disorders include stroke, Alzheimer's, Parkinson's, Huntington's, and various mood disorders such as depression. In many of these cases the locus of deficient activity has been identified and a strategy for treatment involves providing newly functioning networks to the deficient sites. Unless and until it becomes possible to induce neurogenesis in arbitrary brain regions, currently not possible, the delivery of new neurons will be a necessary component of treating these increasingly common afflictions. It is also desirable to induce a functional architecture in such newly-delivered neurons. Even if neurogenesis were to become feasible, it will still be of clinical importance to foster a functional architecture among the new cells.

Although there have been many strategies described in the prior art for alleviating damage to the nervous system through the delivery of new, undamaged neurons, such prior strategies provide no method for programming the implanted neurons for the function they are meant to adopt, or priming them for the electrical environment in which they should participate.

Prior research has considered the use of conductive polymers for neural implants. For example, Schmidt, C. E., et al. in Proceedings of the National Academy of Sciences of the United States of America, 8948-8953 (1997) showed that electrical stimulation of neurons on “blanket” films provides a positive effect as measured by neural outgrowth in cell culture. Research has also looked at conductive polymers for neural implants and has shown that peptides incorporated within the polymer have a beneficial effect. This research has addressed the coating of non-degradable materials (such as gold and silicon) with a conductive polymer but did not teach a free-standing all-polymer, or all-degradable device. See, Cui, X. et al. in Journal of Biomedical Materials Research, 261-72 (2001). Other research has addressed the patterning of pyrrole using “soft lithography” but not for application as a neuroprosthesis. See, Huang, Z. et al. in Synthetic Metals, 1375-1376 (1997) and Jeon, N. L., et al. in Advanced Materials, 946-950 (1999). Tessier, D. et al. in Journal of Biomaterials Science, Polymer Edition, 87-99 (2000) examined the patterning of pyrrole on polyester fabrics for biomedical applications without developing any application in particular. Diaz, A. F. et al., in J. Chem. Soc., Chem. Commun. 635-636 (1979) discusses electrochemical deposition of pyrrole and Warren, L. F. et al. in J. Electrochem. Soc., 101-105 (1987) indirectly discussed the relation between adhesion of the films and composition or deposition conditions. No prior research of which we are aware has addressed the ability to engineer or select the adhesion by altering the composition and/or deposition conditions. Other prior research has shown that mammalian cells will grow on pyrrole films in cell culture conditions. See Wong, J. Y., et al. in Proceedings of the National Academy of Sciences of the United States of America, 3201-3204 (1994). U.S. Pat. No. 6,095,148 relates to neuronal stimulation using electrically conducting polymers in which biomolecules are covalently attached to the conductive polymer. This patent does not relate to implants that are patterned into multiple discrete electrodes nor does it teach the incorporation of biomolecules trapped and released from within the polymer. This prior art patent also does not teach the use of peptides as a dopant.

### SUMMARY OF THE INVENTION

In one aspect, the invention is a method for regenerating function in the nervous system including implanting in a central or peripheral nervous system environment neurons programmed for a selected function in the implant environment. In a preferred embodiment of this aspect of the invention the neurons are programmed using a multi-electrode device such that the neurons are programmed electrically. In another embodiment, the neurons are programmed physically. The multi-electrode device may be adapted to induce synaptic plasticity in the neurons. The neurons may be programmed before or after implantation. In a preferred embodiment the multi-electrode device is degradable in the body. It is also preferred that the multi-electrode device induce changes in functional connectivity of the neurons.

In this aspect of the invention it is also preferred that the multi-electrode device include a pattern of conducting elements and that the conducting elements comprise at least two discrete sites of current or voltage delivery. It is also preferred that the pattern of conducting elements are supported on a conducting or a non-conducting substrate. In another preferred embodiment spatiotemporal patterns of electrical stimulation are delivered to the neurons prior to, during or after implantation to foster enhanced functional integration between the implanted neurons and native neuronal circuits.

In yet another aspect, the invention is an implantable neuronal network construct including a conductive polymer substrate and neurons programmed for a selected function residing on the substrate. The substrate includes a plurality of discrete conducting electrodes. In a preferred embodiment of this aspect of the invention, the substrate is patterned by photo-or-e-beam-lithography, printing, electrodeposition, stamping, direct writing or self assembly. In another embodiment of this aspect of the invention, biomolecules are incorporated into the conductive polymer. Example biomolecules are proteins, antibodies, nerve growth factors, hormones, peptides, inhibitors and anti-inflammatory agents. It is preferred that two or more compounds be incorporated into the conductive polymer. One example is poly(sytrenesulfonate), poly(ethylene glycol) and a peptide. In a preferred embodiment the peptide is a segment of a neurotrophic factor. In another embodiment, the inhibitor is an anti-apoptotic factor or anti-glial factor. A suitable conductive polymer is polypyrrole or biorubber. The compounds incorporated into the conductive polymer or the supporting substrate are selected to control properties such as conductivity, solubility, biocompatibility, inflammation, bioactivity and neuronal plasticity. Peptides may be selected to increase extracellular matrix formation.

In yet another aspect, the invention is a method for making a patterned neuronal tissue construct including harvesting neurons from donor tissue, growing the neurons on a multi-electrode substrate and electrically inducing, using the multi-electrodes, a functional architecture across the neurons.

In yet another aspect, the invention is a method for making a patterned neuronal tissue construct including harvesting neurons from donor tissue and growing the neurons on a polymer substrate having micro-patterned domains to induce physical pattern formation.

Yet another aspect of the invention is a method for manufacturing a multi-electrode device including fabricating at least two electrodes on a conductive template in a manner such that the electrodes can be released from the template. In a preferred embodiment the electrodes are released from the template by dissolution of the template. A suitable template to be dissolved is a metal such as aluminum, copper and titanium. The electrodes may be separated from the template by controlling adhesion between the electrode and the template, thereby allowing a pattern from the template to be removed without damage. Adhesion may be controlled by depositing two or more layers of conductive polymer with chemical or adhesive properties. Adhesion may also be controlled by selecting deposition conditions and dopants to minimize film adhesion.

The electrodes may be deposited onto a conductive substrate which may be patterned by e-beam lithography, printing, stamping, direct writing or self-assembly. It is preferred that the electrodes be released from the template without additional support. In another embodiment of this aspect of the invention the electrodes are degradable and are released from the template with the addition of a supporting layer of non-conductive, degradable material. The non-conductive material is deposited over the degradable electrodes by means of casting, coating or vapor deposition.

The non-conductive degradable material may be attached or deposited in the form of a film, fabric or mesh. Suitable non-conductive degradable materials include PLGA, PLA, HA, biorubber, oxide glasses, and other biocompatible, biodegradable materials known to those skilled in the art. Suitable electrode materials include conjugated polymers, polypyrrole, polythiopene, polyaniline, substituted polyaniline, poly(ethylene dioxythiopene), and polymers with conductive fillers.

The primary technical advantage of our invention over existing conceptions is the use of multi-site electrical stimulation of implanted networks to induce a functional organization compatible with the network's intended purpose. Another advantage is that selecting a biocompatible polymer such as polypyrrole for the implant substrate permits the host nervous system tissue such as brain tissue to more readily accept the substrate and it's supported networks. Further, the use of biodegradable materials such as polypyrrole or poly(glycerol-sebacate) allows the implant substrate to fade from prevalence after successful implantation to make room for complete regeneration and connectivity of cells. Finally, the use of elastomeric material such as poly(glycerol-sebacate) permits a more flexible substrate to be placed in proximity to delicate neural tissue, thereby preventing shearing and insuring that the implants do not further exacerbate damaged neural areas.

### BRIEF DESCRIPTION OF THE DRAWING

The invention is described with reference to the several figures of the drawing, in which,

FIG. 1A is a photomicrograph of neural circuits cultured on glass.

FIG. 1B is a photomicrograph of neural circuits cultured on gold.

FIG. 1C is a graph illustrating spontaneous synaptic activity.

FIG. 1D is a graph illustrating evoked action potentials.

FIG. 1E is a graph illustrating network bursting.

FIG. 2A is a photomicrograph of a neural network integrated with a multi-electrode array of sixty-four elements.

FIG. 2B is a photomicrograph illustrating concurrent visualization of functional synaptic activity.

FIG. 2C is a photomicrograph of neural networks integrated with a sixty-electrode array.

FIG. 2D is a photomicrograph illustrating concurrent visualization of single-cell electrophysiology.

FIG. 3A is a visualization of responses across a network while stimulating a single site (*) to map point-to-point connectivity.

FIG. 3B is a visualization showing a long-lasting enhancement of the site's network influence after a twenty minute training paradigm.

FIG. 3C is a visualization of the decrease of connections from a neighboring site.

FIG. 4 is a photomicrograph showing that polypyrrole supports elaboration of both neurons and glial support cells.

FIG. 5 is a photomicrograph showing the patterned deposition of polypyrrole to generate multiple conductive channels with several-micron resolution.

FIG. 6 is a photomicrograph illustrating a biorubber scaffold with polypyrrole circuitry thereon.

FIG. 7 is a photomicrograph showing polypyrrole chips implanted in rat visual cortex.

FIGS. 8, A, B, C, and D are photomicrographs illustrating integration between existing cortex and the neural implants of the invention.

### DETAILED OF THE PREFERRED EMBODIMENT

As discussed above, the present invention includes a multi-step approach to develop and implant patterned neuronal tissue constructs. These constructs may be based on physical patterning of neurons on polymers or on pre-programmed (electrically patterned) networks of neurons on polymers for integration into the nervous system. We have demonstrated the feasibility of harvesting neurons from donor tissue, growing those neurons on a multi-electrode substrate to electrically induce a functional architecture across the network, or growing those neurons on a polymer substrate that has micro-patterned domains to induce physical pattern formation, and then implanting the neuron-substrate construct into living tissue to replace or augment nervous system function.

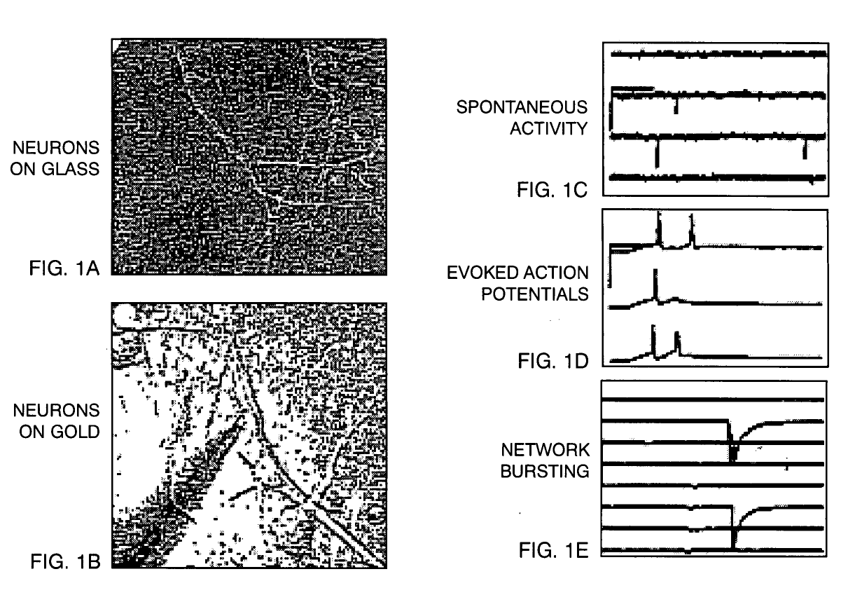

We have made use of a system, established by others, by which neurons can be removed from neonatal animals and grown in-vitro in a manner accessible to observation and electrical stimulation. Working with cortical and hippocampal cells from neonatal rats, we found that our in-vitro system supports cell populations of a similar composition to the original tissue, and that our networks develop endogenous electrical activity with a developmental time course comparable to their in-vivo counterparts. With reference to FIGS. 1A and 1B, we have demonstrated that circuits can be formed on substances such as glass and gold to biopolymers such as polypyrrole in which cells that are initially dissociated from one another extend connections to form a new neuronal network devoid of intentional patterning. The growth can be controlled through the use of electrical stimulation or the patterning of the substrate. FIGS. 1C, D, and E show that the circuits exhibit normal electrical properties such as spontaneous synaptic activity, evoked action potentials, and network bursting respectively.

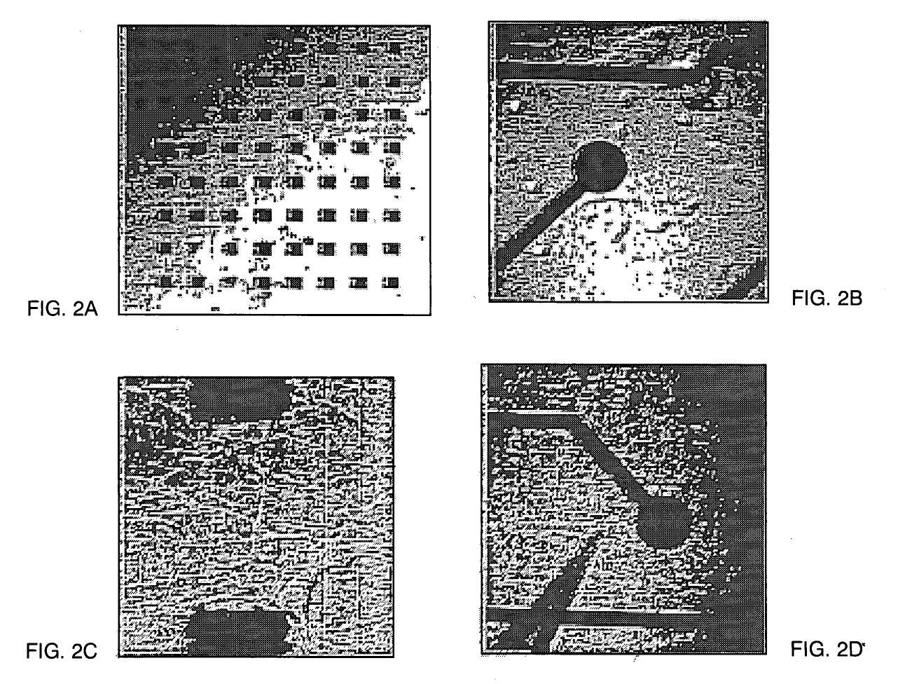

In order to develop a method for bathing our developing circuits in patterned electrical stimulation, it was necessary to interface them with multi-site arrays of electrodes that can be stimulated and recorded independently. We thus applied our culturing techniques illustrated in FIG. 1 to a new substrate: multi-electrode arrays, which are comprised of a glass surface embedded with sixty or sixty-four planer gold electrodes. Our techniques led to sustained growth of healthy cells on this substrate. Reference is made to FIGS. 2A-2D showing neural networks integrated with different multi-electrode array formats and showing concurrent visualization of functional synaptic activity and single-cell electrophysiology.

It was necessary to find a way to localize cell growth to a particular sub-millimeter region of the array since the electrodes themselves only occur in the very center of the glass chip. We confronted this challenge of cell localization by developing a cell adhesion cocktail that was micro-plated to the target region and that would also promote cell health without the usual disruption that neurons experience when grown in small colonies.

Another method for controlling regions of neuronal growth would be to use the polymer substrates themselves to pattern the neurons. We have developed various forms of polypyrrole using different dopants that encourage neuronal growth as well as types of polymer that prevent neural cells from growing. The different characteristics are determined by the dopants that are plated along with the polypyrrole. Various dopants that encourage neuronal growth include but are not limited to brain derived growth factor, neuronal growth factor, sodium salt, and laminin peptides. Other dopants decrease the formation of neuronal networks such as polyaspartic acid and sodium acetate. The combination of these types of polymers allow containment of the neurons to specific sites of the array.

Once we had neurons growing within the small region where electrodes could contact them, we were one step closer to applying patterned stimulation and recording from our populations of cells. We thus connected our arrays to a 64-channel amplifier, and succeeded in interfacing it via data acquisition cards to software that could analyze the spiking information derived from the network. The software is equipped to detect patterns in the cell population's functional connectivity, compare this connectivity to an idealized template, and suggest stimulation patterns that might shift the functional connectivity closer to the target configuration. Finally, to complete the electrical feedback loop to the network of cells, we also connected an 8-channel stimulus generator to the array to excite arbitrary locations of the network in space and time as defined by the computer.

With electronics and software for observing, interpreting, and modifying a neuronal network thus generally in place, we were then interested in the acuity of our recording and stimulation methods: it was vital that our system be equipped with the accuracy to assess precise changes in network connectivity, as well as the precision to focally induce plasticity at specific points in the network. To establish the range of stimulation that would impact a small subset of the network's cells, we performed simultaneous stimulation and recording: injecting current into one electrode while monitoring the impact throughout the network on the other sixty-three electrodes. By virtue of this process we determined the amount of current that was necessary to excite one area of the neuronal network and induce communication between it and other parts of the network.

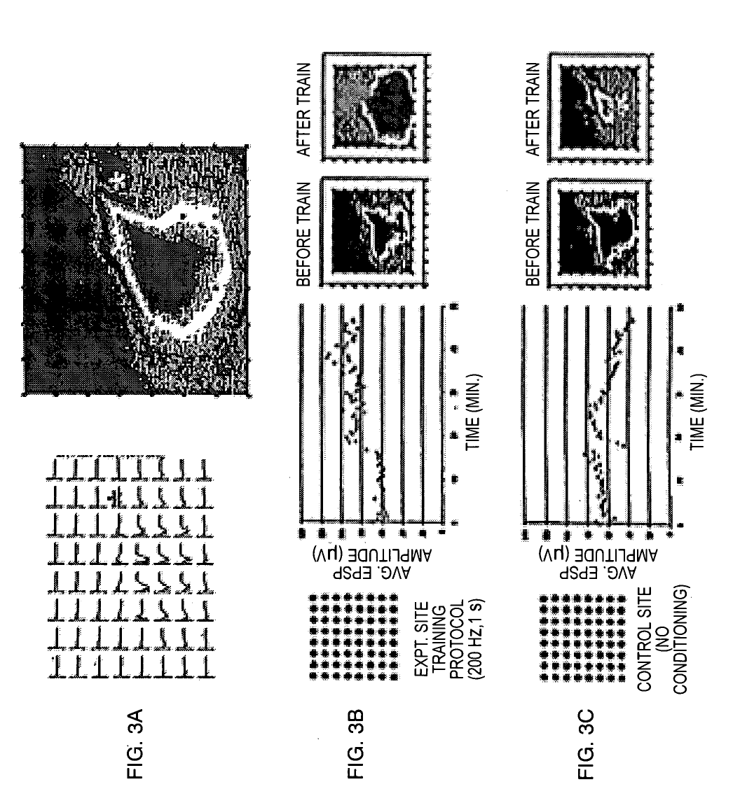

We next undertook reprogramming network connectivity with applied electrical patterns. Neurons are known to change their response properties and revise their connections in response to electrical activity from their neighbors or, artificially, from external devices. In order to demonstrate the feasibility of re-shaping such network connectivity using our multi-electrode arrays, we conducted studies mapping site-to-site connectivity, applying electrical patterns of stimulation to regions of the network, and then mapping site-to-site connectivity again to quantify any changes that may have occurred either locally or across the network. FIGS. 3A-3C illustrate basic reprogramming of network connectivity. FIG. 3A visualizes responses across the network while stimulating a single site marked by the asterisk to map point-to-point connectivity. As shown in FIGS. 3B and C, applying a twenty minute training paradigm at the site marked by the asterisk generates a long lasting enhancement of that site's network influence (FIG. 3B) at the expense of connections from a neighboring site (FIG. 3C).

Protocols that have already proven effective at strengthening or weakening specific subsets of connections are single, high frequency bursts applied to a single site, or paired, “associational” stimulation between dyads of sites. Each of these protocols resulted in an observable strengthening of connections between some regions of the network, and a concomitant weakening of other connections. Responses before and after the stimulation protocols were stable, indicating that we could, in a controlled fashion, alter site-to-site neuronal connectivity to manually re-sculpt or “program” the network. Further, independent control over various electrode access points can allow global patterns of stimulation to be applied that mimic the stimulation patterns occurring endogenously, so that the in-vitro circuits can begin to be prepared for the afferent and recurrent input with which it will eventually integrate.

Multi-electrode arrays comprised of conventional materials will likely be rejected by any host nervous tissue into which it is implanted. It is thus necessary to demonstrate the feasibility of a substrate material that is both capable of providing multi-site stimulation and is biocompatible with respect to brain and other nervous system tissue.

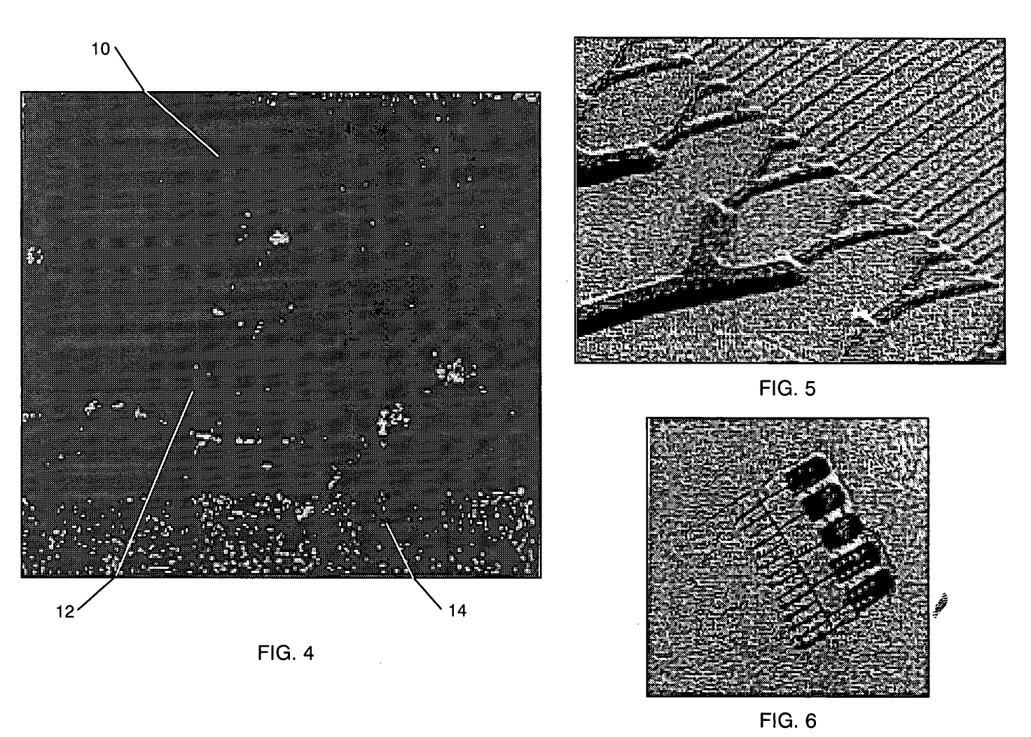

We have demonstrated biocompatibility between neurons and such materials as polypyrrole and poly(glycerol-sebacate) (“biorubber”). FIG. 4 illustrates that polypyrrole can support elaboration of both neurons (cell bodies 10 and extended processes 12) and glial support cells 14. Conductive polymers such as polypyrrole offer the reality of biocompatible conductive channels (“wires”) being formed to mediate applied patterns of electrical stimulation to networks of neurons situated directly above. We have proven the feasibility of controlling the deposition of conductive biopolymer to provide for a fine (less than ten microns wide) circuitry capable of mediating multi-site electrical stimulation. FIG. 5 shows patterned deposition of polypyrrole to generate multiple conductive channels with several-micron resolution. This material can be wired into a biocompatible chip and connected to conventional electronics to mediate the electrical programming of the networks, and then later maintain support for the neuronal networks as the electronics are detached and the chip is implanted. The feasibility of biocompatible wires was an important step in developing a biocompatible substrate capable of delivering programming to neuronal networks.

We have also developed methods to release the polymer substrate from the patterning template to produce a stand-alone polymer microarray. Various types of polypyrrole dopants have been tested to determine which ones have less affinity for the patterning template and can release with greater ease. Using two or more types of polymer can control the conductive polymer release: plating a polymer with low affinity for the substrate first and then plating another form of the polymer with the desired properties. We have also developed a method whereby the conductive polymer can be patterned using one template and then transferred to another substrate. The second substrate, such as poly(glycerol-sebacate), is deposited and then removed along with the adherent conducting polymer. All these methods allow us to produce biodegradable devices that have been released from the original template. These released, biodegradable devices can then be used for neural support and implantation.

Another important consideration is the substrate through which the wires run, which will provide the primary scaffold for the neuronal network during implantation. We have generated such substrates using either non-conductive forms of polypyrrole or, alternatively, biorubber. We have found both polypyrrole and biorubber to be compatible with neurons and have plated biorubber bases with polypyrrole circuitry on top as shown in FIG. 6. Biorubber and other elastomeric compound offer a more flexible substrate that when implanted would help prevent further shearing of damaged areas.

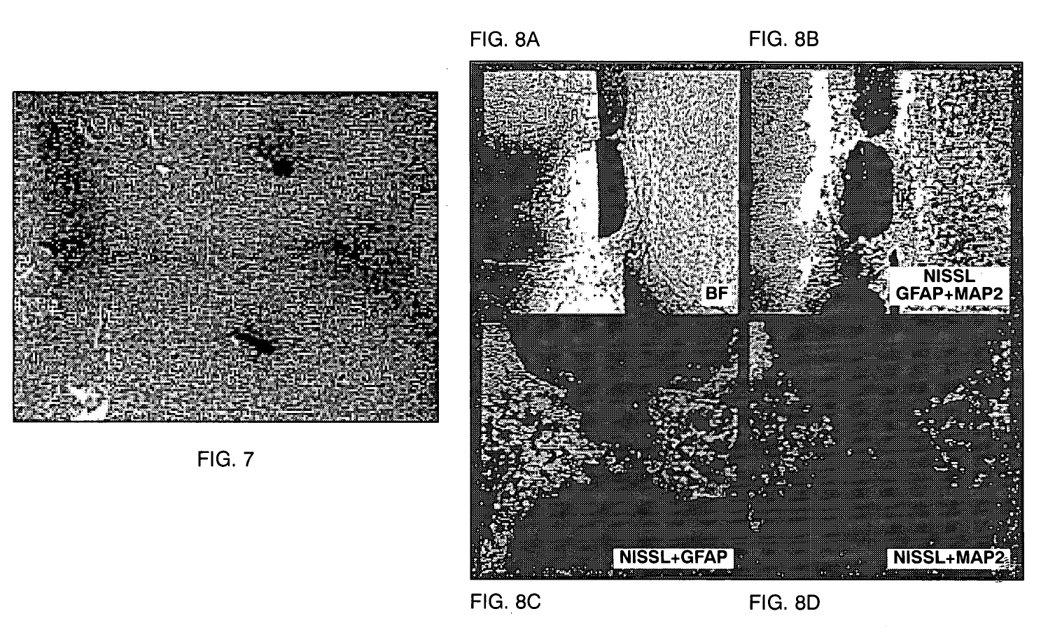

To demonstrate that our biopolymer-based chips are compatible with live brain and can foster integration, we have implanted two-millimeter by three-millimeter versions of the construct into rat visual cortex as shown in FIG. 7. Assessing for normal behavior over 3 to 6 week time points, we then performed post-mortem histology to determine the extent of rejection or integration between the polypyrrole chips and surrounding brain. We found, using immunohistochemistry for the neuronal cell bodies and synaptic connections surrounding the implant site, that surrounding cortex tends to envelop the chips, forming an indistinguishable seal around them, and elaborating processes into the spaces intentionally designed into the chips to assess integration as illustrated in FIG. 8. Efforts are currently under way to demonstrate the same biocompatibility in-vivo for chips based on more elastomeric biopolymers.

We also contemplate incorporation of dopants into the conductive polymer or substrate. Given the flexibility of biopolymers to be integrated with additional materials of biological significance, we have also assayed the efficacy of different substances, comprising various bioactive molecules, for biocompatibility, bio- or non-biological degradation, conductivity and structural properties. Biomolecules that have been tested include proteins, nerve growth factors, hormones, peptides, inhibitors, and hormones that have been incorporated as a mixture into our substrates. By varying the molecules that participate, we find that we can alter the efficacy of integration with biological tissue, as well as the speed at which the substrate degrades to leave room for further integration between host and implanted cells. Multiple dopants have also been incorporated into the polymer matrix to enhance several properties at once. For example, one dopant can address biodegradability while another dopant increases biocompatibility. Being able to form the biomolecule-polymer mixture allows for more control over patterning the neuronal matrix as well as control of the properties of the implants themselves.

It is recognized that modifications and variations of the inventions disclosed herein will be apparent to those skilled in the art and it is intended that all such modifications and variations be included within the scope of the appended claims.

1. Method for regenerating function in the nervous system comprising implanting in a central or peripheral nervous system environment neurons programmed for a select function in the implant environment.

2. The method of claim 1 wherein the neurons are programmed using a device having at least one electrode.

3. The method of claim 2 wherein the device is a multi-electrode device.

4. The method of claim 1 wherein the neurons are programmed electrically.

5. The method of claim 1 wherein the neurons are programmed physically.

6. The method of claim 3 wherein the multi-electrode device induces synaptic plasticity in the neurons.

7. The method of claim 1 wherein the neurons are programmed before implantation

8. The method of claim 1 wherein the neurons are programmed after implantation.

9. The method of claim 2 wherein the device is degradable in the body.

10. The method of claim 3 wherein the multi-electrode device induced changes in functional connectivity of the neurons.

11. The method of claim 3 wherein the multi-electrode device comprises a pattern of conducting elements.

12. The method of claim 11 wherein the conducting elements comprise at least two discrete sites of current or voltage delivery.

13. The method of claim 11 wherein the pattern of conducting elements is supported on a conducting substrate.

14. The method of claim 11 wherein the pattern of conducting elements is supported on a non-conducting substrate.

15. The method of claim 4 wherein spatiotemporal patterns of electrical stimulation are delivered to the neurons prior to, during, or after implantation to foster enhanced functional integration between the implanted neurons and native neuronal circuits.

16. Implantable neuronal network construct comprising:
a conductive polymer substrate; and
neurons programmed for a selected function residing on the substrate.

17. The construct of claim 16 wherein the substrate comprises a plurality of discrete conducting electrodes.

18. The construct of claim 16 wherein the substrate is patterned by photo- or e-beam lithography, printing, electrodeposition, stamping, direct writing or self-assembly.

19. The construct of claim 16 wherein biomolecules are incorporated into the conductive polymer.

20. The construct of claim 19 wherein the biomolecule is a protein.

21. The construct of claim 19 wherein the biomolecule is an antibody.

22. The construct of claim 19 wherein the biomolecule is a nerve growth factor.

23. The construct of claim 19 wherein the biomolecule is a hormone.

24. The construct of claim 19 wherein the biomolecule is a peptide.

25. The construct of claim 19 wherein the biomolecule is an inhibitor.

26. The construct of claim 24 wherein the peptide is a segment of a neurotrophic factor.

27. The construct of claim 25 wherein the inhibitor is anti-apoptotic factor or anti-glial factor.

28. The construct of claim 16 wherein the conductive polymer is polypyrrole.

29. Method for making a patterned neuronal tissue construct comprising: harvesting neurons from donor tissue;
growing the neurons on a multi-electrode substrate; and
electrically inducing, using the multi-electrodes, a functional architecture across the neurons.

30. Method for making a patterned neuronal tissue construct comprising:
harvesting neurons from donor tissue; and
growing the neurons on a polymer substrate having micro-patterned domains to induce physical pattern formation.

31. Method for manufacturing a multi-electrode device comprising:
fabricating at least two electrodes on a conductive template in a manner such that the electrodes can be released from the template.

32. The method of claim 31 wherein the electrodes are released from the template by dissolution of the template.

33. The method of claim 32 wherein the template that is dissolved is a metal.

34. The method of claim 33 wherein the metal is selected from the group consisting of aluminum, copper and titanium.

35. The method of claim 31 wherein the electrodes are separated from the template by controlling adhesion between the electrodes and the template thereby allowing a pattern from the template to be removed without damage.

36. The method of claim 35 wherein adhesion is controlled by depositing two or more layers of conductive polymer with chemical or adhesive properties.

37. The method of claim 35 wherein adhesion is controlled by selecting deposition conditions and dopants to minimize film adhesion.

38. The method of claim 31 or 35 wherein the electrodes are deposited onto a conductive template.

39. The method of claim 38 wherein the conductive template is patterned by e-beam lithography, printing, stamping, direct writing or self-assembly.

40. The method of claim 31 or 35 wherein the electrodes are released from the template without additional support.

41. The method of claim 31 or 35 wherein the electrodes are degradable and are released from the template with the addition of a supporting layer of non-conductive, degradable material.

42. The method of claim 41 wherein the non-conductive material is deposited over the degradable electrodes by means of casting, coating, or vapor deposition.

43. The method of claim 41 wherein the non-conductive degradable material is attached or deposited in the form of a film, fabric or mesh.

44. The method of claim 4.1 wherein the non-conductive degradable material is selected from the group consisting of PLGA, PLA, HA, biorubber, oxide glasses, and other biocompatible, biodegradable materials.

45. The method of claim 31 or 35 wherein the electrodes are selected from the group consisting of conjugated polymers, polypyrrole, polythiopene, polyaniline, substituted polyaniline, poly(ethylene dioxythiopene), and polymers with conductive fillers.

46. The method of claim 45 including the further step that the electrodes are doped with biomolecules selected from the group consisting of proteins, antibodies, nerve growth factors, hormones, peptides, inhibitors, a segment of a neurotrophic factor and an anti-apoptotic factor or antiglial factor.
